(19) United States
(12) Patent Application Publication
(10) Pub. No.: US 2011/0152294 A1
(43) Pub. Date: Jun. 23, 2011

(54) USE OF OSMOLYTES OBTAINED FROM EXTREMOPHILIC BACTERIA FOR THE PRODUCTION OF INHALABLE MEDICAMENTS FOR THE PROPHYLAXIS AND TREATMENT OF PULMONARY AND CARDIOVASCULAR DISEASES AND AN INHALATION DEVICE COMPRISING OSMOLYTE AS ACTIVE AGENT COMPONENT

(76) Inventor: Jean Krutmann, Wegberg (DE)

(21) Appl. No.: 13/038,641

(22) Filed: Mar. 2, 2011

Related U.S. Application Data
(62) Division of application No. 10/563,587, filed on Apr. 18, 2006.

(30) Foreign Application Priority Data
Jul. 7, 2003 (DE) 10330768.0

(51) Int. Cl. A61K 31/505; A61P 9/00; A61P 11/00; A61P 29/00

(52) U.S. Cl. 514/256

(57) ABSTRACT

The invention relates to the use of osmolytes, for example, ectoine, hydroxyectoine, firoin, firoin-A, diglycerol phosphate, cyclic diphosphoglycerate, 1,3-dimannosyl-di-myo-inositol-phosphate (DMIP) and/or diinositol phosphate and their equally effective derivatives and/or pharmacologically acceptable salts thereof, for the combating of pulmonary diseases caused by the effect of airborne particles and/or causally related cardiovascular diseases.

The invention further relates to a dosing device suitable for inhalable medicaments and filled with active agent, the atomizable content of which consisting of an active agent composition comprising at least one osmolyte or its derivatives and/or pharmacologically acceptable salts thereof.

Human health is increasingly impeded by environmental noxa. This is particularly due to air pollution caused by airborne dust which may be fibrous or particulate in nature. Epidemiological investigations have shown that airborne particulates are contributing to the development of pulmonary affections and cardiovascular diseases. In large European cities 60,000 fatalities per year are associated with long-term air pollution. Airborne particulates play a major part in air pollution. Assumptions are that for the time being it will, in fact, not be possible to significantly reduce by filtering measures the airborne particulate exposure, especially that caused by fine and ultrafine airborne particles. On the contrary, it is to be expected that this exposure will even increase considerably. From the URL http://propulmone.ch/Staubpartikel it is known that an individual inhales a minimum of 400,000 m³ of air during its lifetime. With an average particle concentration of 30 μg per m³ of outer air and an assumed 20% proportion of it retained in the lungs approximately 100 particles per day are deposited in each of the 300 millions pulmonary alveoli. In smoker households exposure is expected to be about 20 to 45% higher. The harmful effects to be associated with airborne particles are due to an interaction between these molecules and the human pulmonary tissue. As a result of this, inflammatory and, at times, even malignant pulmonary diseases will be experienced. Suspended particulate matter entering the lungs is thus to be viewed as one of the most significant causes of pulmonary diseases.

Moreover, it is assumed that the exposure to airborne particulate via the lung cells and the biological effects subsequently taking place in these cells must at least be seen as a factor contributing to the pathogenesis of cardiovascular diseases.

It thus follows that in an industrial society there is an ever increasing exposure of the lungs to airborne or suspended particulate which doubtlessly contributes to disease-related fatalities increasing to a degree not to be underestimated. Moreover, it is known from the above mentioned URL that according to recent studies effects harmful to health must even be assumed in the event of suspended particulate concentration hitherto viewed as unobjectionable. Investigations conducted in several large cities revealed that with the daily airborne particulate exposure dose going up by as little as 10 μg/m³ non-accident related fatalities also increased by 0.5 to 1%. Since, as mentioned above, the suspended particulate concentration in the air cannot be reduced effectively and as the use of respiration filters can only be resorted to in exceptional cases, generally and simply applied means have to be looked for by means of which harmful exposure on the lungs can be alleviated and consequential damage kept to a minimum.

As regards people that cannot circumvent being exposed daily to suspended particulate, for example at the workplace, and especially those showing particular sensitivity to airborne particles it is thus of primary importance to develop preventive measures that can be applied on a general basis, simply and at any desired time.

Therefore, it is the objective of the invention to provide pharmaceutical means capable of effectively combating the above described negative effects of airborne particulate on the health of human beings, in particular pulmonary and cardiovascular diseases.

It has been found that pharmaceutical preparations in inhalable form containing one or several osmolytes, the salts thereof and/or their equally effective derivatives, surprisingly, enable an effective prophylaxis to be achieved against the above described diseases as well as treatment of such diseases.

Osmolytes are obtained from extremophilic bacteria. These are extraordinary microorganisms capable of existing and reproducing under the most extreme conditions, e.g. in the presence of extremely high salt concentrations of up to 200 g of sodium chloride per liter and temperatures ranging between 60 and 110° C. Such habitat conditions would cause the immediate death of normal (mesophilic) organisms or would at least lead to an extensive damage of cellular structures. In recent years comprehensive research efforts have therefore been made to identify the biochemical components that are the reason for and bring about the remarkable thermal, chemical and physical stability of the cell structures found in extremophilic organisms.

The high temperature stability of cell structures is—to a remarkable extent—due to low-molecular organic substances present in the intracellular environment which are known as osmolytes or compatible solutes. Osmolytes found in extremophilic microorganisms are not produced by human or animal cells.

Recently, various newer osmolytes have been identified for the first time in extremophilic microorganisms. These include, for example, ectoine, hydroxyectoine, firoin, firoin-A, diglycerolphosphate, cyclic diphosphoglycerate, diinositol phosphate, and 1,3 dimannosyl di-myo-inositol phosphate (DMIP). All of them are won from extremophilic microorganisms, then refined and cleaned (refer to EP-A 94 903 874; EP-A 98 121 243; DE-A 100 47 444), thus forming a known group of low-molecular substances offering protection for otherwise sensitive cells. In some cases it could be shown that these compounds contributed to the protection of skin cells against external stress conditions such as heat and dryness in the field of cosmetics (refer to U.S. Pat. No. 6,267,973). It has occasionally been proposed to make use of topical pharmaceutical products aimed at protecting the skin against externally induced stress or treating illnesses caused by the enzymatic decomposition of tissue structures (refer to DE-A 100 06 578). Aside from other diseases of general nature diseases of the immune system, autoimmune related diseases, inflammatory processes as well as acute and chronic inflammations were mentioned in that context.

DE-A 198 34 816 which also proposes the use of osmolytes relates to cosmetic formulations offering skin protection against UV radiation and said products should, moreover, have a stabilizing effect on the nucleic acids of human skin cells. The osmolyte ectoine was also employed as moisturizer in cosmetic preparations with the aim of protecting human skin against the detrimental effects of ultraviolet solar radiation (EP-A 19 990 941).

The production of a pharmaceutical preparation intended for the general treatment of skin diseases by means of osmolytes, in particular ectoine or hydroxyectoine, is known from EP-A 0 887 418 which was filed by the applicant Bitop AG itself. It has been assumed in that context that these agents are conducive to the stabilization of enzymes and other biomolecules and therefore can contribute to the stabilization of denaturizing conditions.

In laid-open patent applications DE-A 199 33 460, DE-A 199 33 461, DE-A 199 33 463 and DE-A 199 33 466 it has been proposed due to their antioxidative effect to use ectoines as free-radical scavengers and in this way protect the skin, especially against ageing accelerated and intensified due to solar radiation. Moreover, undesirable skin states resulting from oxidative phenomena should also be avoided in this manner. Based on premises similar to those proposed in the publications last referred to WO 01/72287 describes the use of ectoines in conjunction with the treatment of UV induced immunosuppression.

Until now it has not been found in which way osmolytes act on tissue other than that of the skin. Even the external application of hydroxyectoine on the cornea and iris of the eyes of rabbits (refer to Heusener report No. T14952 of Apr. 6, 2001) had caused initial irritation and sensitization of the conjunctiva (redness, chemosis and discharge) which, however, disappeared afterwards but, nevertheless, were clear indications to those skilled in the art that incompatibility was to be expected in the case of more sensitive tissue surfaces. In view of these findings skilled persons certainly did not see any reason to bring osmolytes in contact with human lung tissue: although being basically natural active agents they were nevertheless conspicuous substances being “nonnaturally” produced as agents extraneous to the body. Apparently, those skilled in the art have not at all taken into consideration so far that osmolytes might be applied to tissue inside the body, in particular to the highly sensitive lung or bronchial tissue.

Surprisingly, it has now been determined that osmolytes are not only well tolerated by human bronchial and lung tissue including pulmonary alveoli but, unexpectedly, have an excellent prophylactic effect counteracting the noxious influence of suspended particulate irrespective of the nature of such airborne particles. They are also suitable for the treatment of diseases causally originating through such effects.

It is thus possible by an appropriate prophylaxis and/or treatment with suitably dosed inhalation preparations containing osmolytes as active agents to effectively combat not only the generally known and hereinbefore described diseases of the lungs but, moreover, the cardiovascular diseases associated with and originating from them.

The invention relates to the use of osmolytes as well as the equally effective derivatives and/or pharmacologically compatible salts thereof for the combating of diseases caused by the effects of suspended particulate on the lung tissue and/or the cardiovascular diseases that are causally related with them.

The invention further relates to the use of osmolytes as well as their equally effective derivatives and/or pharmacologically compatible salts thereof for the production of pharmaceutical preparations in inhalable form aimed at combating diseases caused by the effects of suspended particulate on the lung tissue and/or the cardiovascular diseases that are causally related with them.

Another objective of the invention relates to an inhalation device filled with active agent, the atomizable solid or liquid contents of which consisting of an active agent composition comprising at least one osmolyte or its derivatives and/or pharmacologically acceptable salts thereof.

It thus follows that when referring to ‘combating’ of lung diseases it is understood and the invention provides that this applies to both the prophylaxis for healthy persons and the treatment of people already suffering from symptoms caused by the effects of suspended particulate exposure.

Some of the active agents according to the invention are weak bases or acids and for that reason may, in some instances even preferably, be employed in their pharmacologically most compatible neutral salt form.

Pharmacologically compatible salts embrace alkaline or alkaline-earth salts, in particular the salts of potassium, sodium, magnesium and calcium but also salts with organic bases such as, for example, with non-toxic aliphatic or aromatic amines.

Should nitrogen atoms be present in the active agent molecule with the basic nature being predominant, salts with pharmacologically unobjectionable organic or inorganic acids are formed such as for example acetic acid, citric acid, tartaric acid, mandelic acid, malic acid, lactic acid, hydrochloric acid, hydrobromic acid, sulfuric acid or phosphoric acid.

Equally effective derivatives are compounds that in comparison to the basic structures of the osmolytes mentioned hereinbefore show structural differences, in particular of the functional groups and substituents, but have equivalent or similar effects within the scope of the invention. In the event of hydroxyectoine for example relevant alkoxyl groups may form from the hydroxyl group with saturated or unsaturated, straight-chain or branched C¹ to C⁴ alkyl groups. With C¹ to C⁴ carboxylic acids relevant esters are formed. From the carboxyl group amides form which in turn may comprise saturated or unsaturated, straight-chained or branched C¹ to C⁴ alkyl groups attached to the nitrogen atom. Using relevant C¹ to C⁴ alcohols effective esters are obtained. The carboxylate group may thus be substituted by a carbonyl, sulfonyl or sulfonylate group. Relevant modifications of the other osmolytes mentioned may be brought about in an analogous manner with the effect being maintained or even enhanced.

Preferred osmolytes are ectoine, hydroxyectoine as well as their derivatives and salts having similar effects. Also preferred are combination preparations containing both active agents coexisting and including further active agents if thought expedient.

Generally speaking, according to the invention the active agents or combinations thereof, including further active agents if thought expedient, may be processed in a known manner to obtain inhalable medicaments making use of auxiliary substances and additives pharmacologically unobjectionable and usually applied in inhalation therapy.

Since osmolytes are easily dissolved in water such additives in the event of inhalable liquid preparations primarily consist of sterile water to which, as the case may be, further solvents, stabilizers, preservation agents or solutizers are added.

Systems of matter comprising solid and/or liquid particles finely dispersed in a gas are termed aerosols. Active agents containing liquids often in the form of solutions are usually atomized as aerosols in a known manner.

Most expediently, solids mixtures are applied by means of so-called powder inhalers via which these solids can be made available for inhalation. Various types of devices are available for administering the active agents including, inter alia, Spinhaler, Diskhaler, Turbohaler, Rotahaler or Aerolizer which are distinguishable as to their different spraying systems or mechanisms.

Therefore, the object of the invention also relates to medical products which serve the application purposes proposed by the invention.

According to the invention it is basically recommendable to limit the amount of auxiliary substances in the inhalant to a minimum and only use for powder inhalers carrier substances—such as, for example, micronized lactose—which are easily resorbed and non-irritating. Appropriately micronized solids are especially suitable as carrier substances when they contain the active agents in adsorbed or absorbed form. In recent times, this form of inhalation therapy has become more and more accepted. Newer solids inhalers enable applications to be implemented in an especially simple and safe manner.

Aside from the active agents proposed by the present invention further active agents suitable for treatment may be added as necessary. This includes for example antiasthmatics, broncholytics or expectorants.

Liquid dosing aerosols can be employed in conjunction with customary propellants such as, for example, the CFC propellants dichlorodifluoromethan, trichlorofluoromethan or cryofluorane. Preferred here are non-halogenated propellants such as propane or butane or compressed non-toxic gases such as nitrogen, carbon dioxide or dinitrogen monoxide.

The active agents according to the invention may be processed to obtain practically all inhalable preparation forms. Such preparation forms are, for example, solutions, liquid/solids dispersions, solids/solids dispersions, suspensions and emulsions.

The concentration of the active agents ranges between 0.005 and 20 percent by weight based on the weight of the carrier material employed. Preferred is a range between 0.05 and 2 percent by weight.

The following examples serve to elucidate the invention but are in no way whatsoever meant to limit its scope.

### EXAMPLE 1

Compressed gas inhalant:

| | Percentage |
|---|---|
| Constituents | by weight |
| Water | 97.0 |
| Ectoine | 0.5 |
| Hydroxyectoine | 0.5 |
| Preservation agent | 0.2 |
| Nitrogen propellant | |

### EXAMPLE 2

Powder inhalant:

| | Percentage |
|---|---|
| Constituents | by weight |
| Microcrystalline lactose | 99.4 |
| Ectoine | 0.3 |
| Hydroxyectoine | 0.3 |

1.-10. (canceled)

11. A method of treatment of diseases caused by the effects of suspended particulate on lung tissue and/or the cardiovascular diseases related thereto comprising administering an effective amount of at least one osmolyte selected from ectoine, hydroxyectoine or a pharmacologically compatible salt thereof to a patient.

12. A method for the production of inhalable pharmaceutical preparations aimed at combating of diseases caused by the effects of suspended particulate on lung tissue and/or the cardiovascular diseases related thereto comprising using an effective amount of at least one osmolyte selected from ectoine hydroxyectoine or a pharmacologically compatible salt thereof.

13. A method according to claim 11, characterized in that further active agents are added to the preparation.

14. A method according to claim 12, characterized in that further active agents are added to the preparation.

15. The method of claim 11, wherein the diseases are inflammatory diseases.

16. The method of claim 12, wherein the diseases are inflammatory diseases.

17. A method for the production of inhalable pharmaceutical preparations aimed at combating diseases caused by the effect of suspended particulate on lung tissue and/or cardiovascular diseases related thereto, comprising providing in an inhalation device an effective amount of at least one osmolyte selected from ectoine, hydroxyectoine or a pharmacologically compatible salt thereof as an active agent in form of an atomazible solid or liquid.

18. The method of claim 17, wherein the diseases are inflammatory diseases.
